(19) United States
(12) Patent Application Publication
(10) Pub. No.: US 2012/0129825 A1
(43) Pub. Date: May 24, 2012

(54) METHOD FOR ON-DEMAND CONTRACEPTION USING LEVONORGESTREL OR NORGESTREL

(76) Inventors: Andre Ulmann, Paris (FR); Erin Gainer, Paris (FR); Elizabeth Gray Raymond, New York, NY (US)

(21) Appl. No.: 13/264,346

(22) PCT Filed: Apr. 13, 2010
(86) PCT No.: PCT/EP2010/054813

§ 371 Date: Jan. 30, 2012

Related U.S. Application Data
(60) Provisional application No. 61/169,158, filed on Apr. 14, 2009.

(51) Int. Cl. A61K 31/567; A61P 15/18

(52) U.S. Cl. 514/179

(57) ABSTRACT

The invention relates to a method for contraception, which method comprises on-demand administering levonorgestrel or norgestrel in a woman, within 24 hours before an intercourse.

The present invention relates to methods for on-demand contraception in a woman, especially women who do not have a regular sexual activity.

### TECHNICAL BACKGROUND OF THE INVENTION

Hormonal contraception is considered the most reliable method of reversible contraception today. It requires the continuous taking of pills, generally daily, regardless of the frequency of intercourses. For women with infrequent sexual intercourse, however, preparations that are dependent on coitus and thus can be taken less often, with reduced exposure to the effective ingredients, would be more advantageous. Although recognized for a long time (Canzler et al, Zbl. Gynäkol, 1984, 106:1182-1191), the need for such on-demand contraception remains unmet (Aitken et al, Contraception, 2008, 78:S28-S35).

Women are actually creating such methods themselves out of existing products. In Ghana, a study conducted in 2003 reported that women were using norethindrone tablets, marketed for treatment of various gynecologic problems, as an “on demand” oral contraceptive. More recently, anecdotal reports and data collected by colleagues at Family Health International indicate that women in other parts of Africa and elsewhere are deliberately using emergency contraceptive pills in this manner.

Although oral methods do not provide protection against sexually transmitted infections, studies conducted several decades ago reported that various doses of levonorgestrel used as a regular postcoital contraceptive may provide protection with an efficacy comparable to the overall efficacy of condoms and other barrier methods in typical use (United Nations Development Programme/United Nations Population Fund/World Health Organization/World Bank Special Programme of Research, Development and Research Training in Human Reproduction, Task Force on Post-Ovulatory Methods of Fertility Regulation. Efficacy and side effects of immediate postcoital levonorgestrel used repeatedly for contraception. Contraception 2000;61:303-8). It was further proposed to evaluate whether a single vaginal administration of levonorgestrel gel prior to intercourse would interfere with the ovulatory process. (Brache et al, Contraception, 2007; 76:111-116).

### SUMMARY OF THE INVENTION

The invention provides a method for contraception, which method comprises on-demand administering more than 0.50 mg levonorgestrel or norgestrel in a woman, within 24 hours before an intercourse, preferably by enteral or transdermal route.

Another subject of the invention is a method for on-demand contraception, which method comprises vaginally administering levonorgestrel or norgestrel in a woman, within 24 hours before an intercourse.

In a preferred embodiment, such method comprises inserting a vaginal ring that releases levonorgestrel or norgestrel in a woman, within 24 hours before an intercourse.

### DETAILED DESCRIPTION OF THE INVENTION:

The inventors propose that levonorgestrel or norgestrel could be used as a contraceptive which could be taken on-demand, rather than everyday as any hormonal contraceptive pill.

### Levonorgestrel And Norgestrel

Levonorgestrel is D-(−)-ETHYL-13BETA ETHYNYL-17ALPHA HYDROXY-17 GONENE-4 ONE-3.

Norgestrel (or DL-norgestrel) is (8R,9S,10R,13S,14S,17S)-13-ethyl-17-ethynyl-17-hydroxy-1,2,6,7,8,9,10, 11,12,14,15,16-dodecahydrocyclopenta[a]phenanthren-3-one.

Levonorgestrel and norgestrel have been developed for regular contraception, as well as for postcoital emergency contraception.

### On-Demand

The subject or patient can be any human female. The invention provides an “on-demand contraception”, which means that the woman may take levonorgestrel or norgestrel at any time when needed, i.e. when an intercourse is expected.

Preferably, the woman does not use any other hormonal contraceptive medication. In another preferred embodiment, the subject does not use any protection (condom, uterine device, spermicide, etc). Preferably no contraceptive is used after the intercourse.

The invention provides a method of contraception, which method comprises discontinuously administering levonorgestrel or norgestrel in a woman before an intercourse.

According to the invention, the woman will not use levonorgestrel or norgestrel on a daily basis, and preferably not more than ten days in a row, preferably not more than nine, eight, seven, six, five, four, three or two days in a row. Preferably levonorgestrel or norgestrel is not administered more than four days, three or two days in a row.

### Routes of Administration

Levonorgestrel or norgestrel may be administered by any convenient route, including oral, buccal, sublingual, parenteral, transdermal, vaginal, rectal, etc. Preferably levonorgestrel or norgestrel may be administered by enteral (including oral, buccal, or sublingual route) or transdermal route.

For a brief review of present methods for drug delivery, see, Langer, Science 249:1527-1533 (1990), which is incorporated herein by reference. Methods for preparing administrable compounds are known or are apparent to those skilled in the art and are described in more detail in, for example, Remington's Pharmaceutical Science, 17th Ed., Mack Publishing Company, Easton, Pa. (1985), which is incorporated herein by reference, and which is hereinafter referred to as “Remington.”

Unit dosages of immediate-release formulations are preferred.

For solid compositions, conventional nontoxic solid carriers may be used which include, for example, pharmaceutical grades of mannitol, lactose, starch, magnesium stearate, sodium saccharine, talcum, cellulose, glucose, sucrose. For oral administration, a pharmaceutically acceptable nontoxic composition is formed by incorporating any of the normally employed excipients, such as those carriers previously listed.

Oral solid dosage forms are preferentially compressed tablets or capsules. Compressed tablets may contain diluents to increase the bulk of levonorgestrel or norgestrel so that production of a compressed tablet of practical size is possible. Binders, which are agents which impart cohesive qualities to powdered materials may be also necessary. Povidone, starch, gelatin, sugars such as lactose or dextrose, and natural and synthetic gums may be used. Disintegrants are generally necessary in the tablets to facilitate break-up of the tablet. Disintegrants include starches, clays, celluloses, algins, gums and crosslinked polymers. Lastly small amounts of materials known as lubricants and glidants are included in the tablets to prevent adhesion of the tablet material to surfaces in the manufacturing process and to improve the flow characteristics of the powder material during manufacture. Colloidal silicon dioxide is most commonly used as a glidant and compounds such as talc, magnesium stearate or stearic acids are most commonly used as lubricants. Procedures for the production and manufacture of compressed tablets are well known by those skilled in the art (See Remington).

Capsules are solid dosage forms using preferentially either a hard or soft gelatin shell as a container for the mixture of levonorgestrel or norgestrel and inert ingredients. Procedures for production and manufacture of hard gelatin and soft elastic capsules are well known in the art (See Remington).

Buccal or sublingual forms or devices are also useful.

Transdermal delivery systems comprising a penetration enhancer and an occlusive backing are of use to deliver levonorgestrel or norgestrel. Examples of penetration enhancers include dimethyl sulfoxide, dimethyl acetamide and dimethylformamide. Transcutenous gels may be advantageous too. Examples of such gels are described e.g. in U.S. Pat. No. 5,904,931.

Levonorgestrel or norgestrel may be administered by vaginal route, for instance in the form of a gel or a vaginal device.

In another embodiment, the method of the invention comprises on-demand inserting a vaginal device, such as a vaginal ring that releases levonorgestrel or norgestrel, in a woman, within 24 hours before an intercourse.

The vaginal ring usually comprises a synthetic polymer, which can be a silicone elastomer or a non-silicone resin. In a particular embodiment the ring comprises a polymer core, surrounded by an outer polymer layer comprising levonorgestrel or norgestrel. In a particular embodiment, the ring is as described in international patent application WO2006/010097.

Preferably, the vaginal ring is removed within 12 hours after the intercourse. Most preferably, the vaginal ring is maintained in place for a minimum of 6 to 12 hours.

The oral route is preferred. Other routes of administration can be suitable in comparison with oral routes using blood levels to provide clinical success.

### Dosages

The unit dosage of levonorgestrel or norgestrel is more than 0.50 mg, preferably between 0.50 and 1.50 mg, preferably between 0.75 and 1.50 mg, preferably 0.75 mg, especially when administered by the enteral or transdermal route. Preferably the unit dosage of levonorgestrel or norgestrel is to be administered orally.

### Regimen

Levonorgestrel or norgestrel can be administered within 24 hours before an intercourse, preferably within 12 h before the intercourse, more preferably within 6 h before the intercourse, preferably within 4 h, preferably within 2 h, still more preferably within 1 h before the intercourse.

Levonorgestrel or norgestrel can be administered within 24 hours before an intercourse, and the same woman can also take levonorgestrel or norgestrel after the intercourse, preferably within 1 h, also.

In a most preferred embodiment, levonorgestrel or norgestrel is administered in the form of a tablet comprising 0.75 mg levonorgestrel or norgestrel, within 1 h before the intercourse, preferably in a woman who does not use any contraceptive after the intercourse, most preferably a woman who does not use any other contraceptive.

1. A method for contraception, which method comprises on-demand administering more than 0.50 mg levonorgestrel or norgestrel in a woman, within 24 hours before an intercourse.

2. The method of claim 1, wherein levonorgestrel or norgestrel is administered at a dosage of between 0.75 and 1.50 mg.

3. The method of claim 1, wherein levonorgestrel or norgestrel is administered at a dosage of 0.75 mg.

4. The method of claim 1, wherein levonorgestrel or norgestrel is administered within 12 h before the intercourse.

5. The method of claim 1, wherein levonorgestrel or norgestrel is administered within 6 h before the intercourse.

6. The method of claim 1, wherein levonorgestrel or norgestrel is administered within 1 h before the intercourse.

7. The method of claim 1, wherein levonorgestrel or norgestrel is administered by enteral or transdermal route.

8. The method of claim 7, wherein levonorgestrel or norgestrel is administered orally.

9. The method of claim 8, wherein levonorgestrel or norgestrel is administered in the form of a tablet.

10. The method of claim 7, wherein levonorgestrel or norgestrel is administered in the form of a transcutaneous gel.

11. The method of claim 1, wherein no contraceptive is used after the intercourse.

12. The method of claim 10, wherein levonorgestrel or norgestrel is administered in the form of a tablet comprising 0.75 mg levonorgestrel or norgestrel, within 1 h before the intercourse.

13. The method of claim 1, comprising administering levonorgestrel or norgestrel before and after the intercourse.

14. A method for on-demand contraception, which method comprises vaginally administering levonorgestrel or norgestrel in a woman, within 24 hours before an intercourse.

15. The method of claim 14, which comprises on-demand inserting a vaginal ring that releases levonorgestrel or norgestrel in a woman, within 24 hours before an intercourse.

16. The method of claim 15, wherein the vaginal ring is removed within 12 hours after the intercourse.
